(19) United States
(12) Patent Application Publication
(10) Pub. No.: US 2016/0310679 A1
(43) Pub. Date: Oct. 27, 2016

(54) BANDAGE HAVING INJECTION PORT AND STERILZATION INDICATOR

(71) Applicant: Frederick Hirth, New River, AZ (US)

(72) Inventor: Frederick Hirth, New River, AZ (US)

(21) Appl. No.: 14/693,768

(22) Filed: Apr. 22, 2015

(51) Int. Cl. A61M 5/42; A61F 13/00; A61F 13/02

(52) U.S. Cl. CPC A61M 5/427; A61F 13/024; A61F 13/00059

(57) ABSTRACT

A bandage has a first section having a first section adhesive backing formed on a rear surface of the first section. An opening is formed through a central area of the first section. An indicator is formed on a front surface of the first section showing when an area around and in the opening has been sterilized. A cover member is attached to the first section closing the opening

### TECHNICAL FIELD

The present application generally relates to bandages, and more specifically, to a bandage having an opening for injection of a needle, a sterile cover and a visual indicator to show when an area around the opening has been sterilized.

### BACKGROUND

When applying vaccinations, drawing blood or performing other procedures where a needle may be inserted into patient, proper sterilization techniques should be followed. Proper sterilization techniques generally require one to sterilize the injection site and place a bandage where the injection took place. When sterilizing the injection site, a medical professional generally wipes the injection site and surrounding area with an alcohol swab and allows then allows the alcohol to dry.

Unfortunately, many medical professionals fail to follow proper sterilization techniques. Many medical professionals either fail to properly sterilize the injection area on the patient or may have even forgotten the exact area on the patient where the medical professional sterilized. As a result, the injection site may become infected.

Therefore, it would be desirable to provide a system and method that overcome the above identified concerns, as well as additional challenges which will become apparent from the disclosure set forth below.

### SUMMARY

In accordance with one aspect of the present application, a bandage is disclosed. The bandage has a first section having a first section adhesive backing formed on a rear surface of the first section. An opening is formed through a central area of the first section. An indicator is formed on a front surface of the first section showing when an area around and in the opening has been sterilized. A cover member is attached to the first section closing the opening.

In accordance with one aspect of the present application, a bandage is disclosed. The bandage has a first section having a first section adhesive backing formed on a rear surface of the first section. An opening is formed through a central area of the first section. An indicator is formed on a front surface of the first section showing when an area around and in the opening has been sterilized. A cover member is attached to the first section closing the opening. A strip attaches the first section to the cover member. The strip attaching the first section to the cover member forms a tab member when the cover member is positioned over the opening to lift and remove the bandage.

In accordance with one aspect of the present application, a bandage is disclosed. The bandage has a first section. An opening is formed through a central area of the first section. A first section adhesive material is applied to a rear section of the first section. A first section backing is applied to the first section adhesive material. An indicator is formed on a front surface of the first section showing when an area around and in the opening has been sterilized. A cover member is attached to the first section closing the opening. A pad is attached to a rear surface of the cover member. A cover adhesive is applied around a perimeter of the pad. A cover backing is applied to the cover adhesive. A strip attaches the first section to the cover member. The strip attaches the first section to the cover member forming a tab member when the cover member is positioned over the opening to lift and remove the bandage.

### BRIEF DESCRIPTION OF THE DRAWINGS

The present application is further detailed with respect to the following drawings. These figures are not intended to limit the scope of the present application but rather illustrate certain attributes thereof.

FIG. 1 is a top perspective view of an exemplary embodiment of a bandage having an injection port and sterilization indicator according to one aspect of the present application; and

FIG. 2 is bottom perspective view of the exemplary embodiment of the bandage shown in FIG. 1 having an injection port and sterilization indicator according to one aspect of the present application;

FIG. 3 is a top perspective view of an exemplary embodiment of a bandage having an injection port and sterilization indicator according to one aspect of the present application

FIG. 4 is a bottom perspective view of the exemplary embodiment of the bandage shown in FIG. 3 having an injection port and sterilization indicator according to one aspect of the present application.

### DESCRIPTION OF THE APPLICATION

The description set forth below in connection with the appended drawings is intended as a description of presently preferred embodiments of the disclosure and is not intended to represent the only forms in which the present disclosure can be constructed and/or utilized. The description sets forth the functions and the sequence of steps for constructing and operating the disclosure in connection with the illustrated embodiments. It is to be understood, however, that the same or equivalent functions and sequences can be accomplished by different embodiments that are also intended to be encompassed within the spirit and scope of this disclosure

Embodiments of the exemplary system and method provide a bandage having an injection port for indicating an area on a body of a patient for insertion of a needle for vaccination or other medical reasons. The injection port may show and remind a medical professional the area that needs to be cleaned with an alcohol wipe. The bandage may have an indicator which shows when the injection area has been properly sterilized. The bandage may have other features as described below.

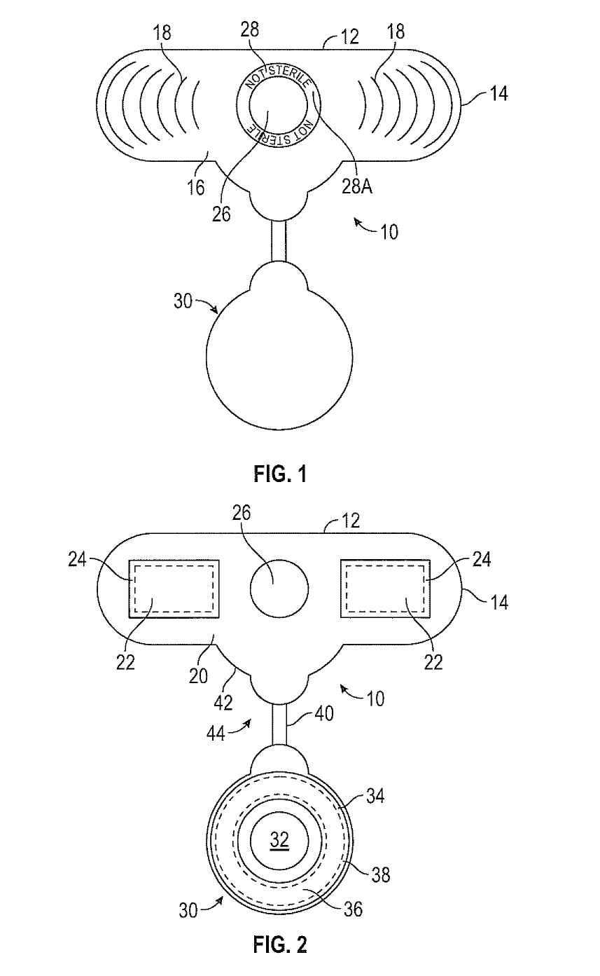

Referring to FIGS. 1-2, one exemplary embodiment of a bandage 10 may be seen. The bandage 10 may have a first section 12. In the present embodiment, the first section 12 may be rectangular in shape having rounded edges 14. However, this is given as an example and should not be seen in a limiting manner. The first section 12 may come in other shapes and/or sizes without departing from the spirit and scope of the present invention.

The first section 12 may be formed out a material that provides unidirectional elasticity and which “lifts” the skin of the patient thereby increasing blood flow and circulation. This may lead to less pressure on the nociceptors, or pain detectors, of the patient. This may theoretically decreases pain from injection of as needle as shown below. In accordance with one embodiment, the first section 12 may be formed out elastic therapeutic tape like kinesiology tape. This type of material is a thin, elastic cotton tape that can stretch up to 140% of its original length. As a result, if the tape is applied to a patient on a stretch greater than its normal length, the tape may “recoil” after being applied and therefore create a pulling “lifting” force on the skin.

The first section 12 may have a top surface 16. The top surface 16 may have a plurality of grip members 18 formed on opposing ends of the top surface 16. The grip members 18 may be formed of a rough texture and/or raised members, or the like. The grip members 18 may be used to grab and pinch the skin of the wearer of the bandage 10. When a medical professional performs an immunization, the medical professional generally “pinches” the skin of the patient with the thumb and index finger of the non-immunizing hand. This may allow for less movement of the patient's skin and allows for a better injection. The grip members 18 may be used to allow the medical professional to obtain a firmer grip on the patient's skin while also reducing the amount of contact between the medical professional and the patient's field of injection. This may reduce contamination and increasing sterility.

The first section 12 may have a bottom surface 20. The bottom surface 20 may have an adhesive member 22 formed on opposing ends thereof. The adhesive members 22 may be used to attach the bandage 10 to the skin of the patient. A backing 24 may be applied over the adhesive members 22. The backing 24 may be removed before attaching the bandage 10 to the skin of the patient.

An opening 26 may be formed through the first section 12 of bandage 10. The opening 26 may be formed through a central area of the first section 12. The opening 26 may be used to indicate an injection area for administering a vaccination or insertion of a needle for other medical purposes into the patient. The opening 26 may remind the medical professional the area that needs to be and/or was sterilized. Without this indicator, the medical professional may swab and forget the exact area that was sterilized.

The area around the opening 26 may have an indicator 28 to show when the area in and around the opening 26 has been sterilized. The indicator 28 may be a chemical that indicates when the area in and around the opening 26 has been sterilized. In accordance with one embodiment, chromic acid, may be applied into the top layer of the area around the opening 26. The chemical reaction between the isopropyl alcohol that is impregnated in the alcohol swabs and the chromic acid turns the field around the opening 26 green. This may let the medical professional know that the injection site/field has been sterilized. Alternatively, the indicator 28 may be indicia 28A formed around the opening 26. The indicia 28A may be text such as “NOT STERILE” or the like, encircling the opening 26. When the isopropyl alcohol saturated alcohol pad is applied in and around the opening 26, the wording, “NOT STERILE” may change colors and/or may evaporate and disappear. This may remind the medical professional that the area in and around the opening 26 has or has not been swabbed prior to injection.

The bandage 10 may have a cover member 30. The cover member 30 may have a pad 32 formed on a first surface 34. The pad 32 may be formed of cotton gauze or like material. An adhesive 36 may be formed around a perimeter of the cover member 30. The adhesive 36 may be used to attach the cover member 30 over the opening 26. A backing 38 may be applied on the adhesive 36. The backing 38 may be removed from the adhesive 36 when the adhesive 36 may be used to attach the cover member 30 over the opening 26.

A retaining strip 40 may be used to attach the cover member 30 to the first section 12. A tab member 42 may extend from the first section 12. The retaining strip 40 may be attached to the tab member 42 and the cover member 30. The tab member 42 and the retaining strip 40 may form a “pull-tab” 44. The “pull-tab” 44 may be used for removing the bandage 10 by lifting on the “pull-tab” 44 up and away from the skin of the patient.

The bandage 10 may be used for reasons other than injection of a needle for medical purposes. For example, the bandage 10 may be used for other reasons such as warts, scabs, moles or insect bites where applications of medicaments may be necessary or desired. The bandage may be used by medical professionals to isolate skin areas where surgical procedures may have occurred, i.e. biopsies, lances, skin tag removal and the like. This may also include pre-biopsies, pre-lances, etc.

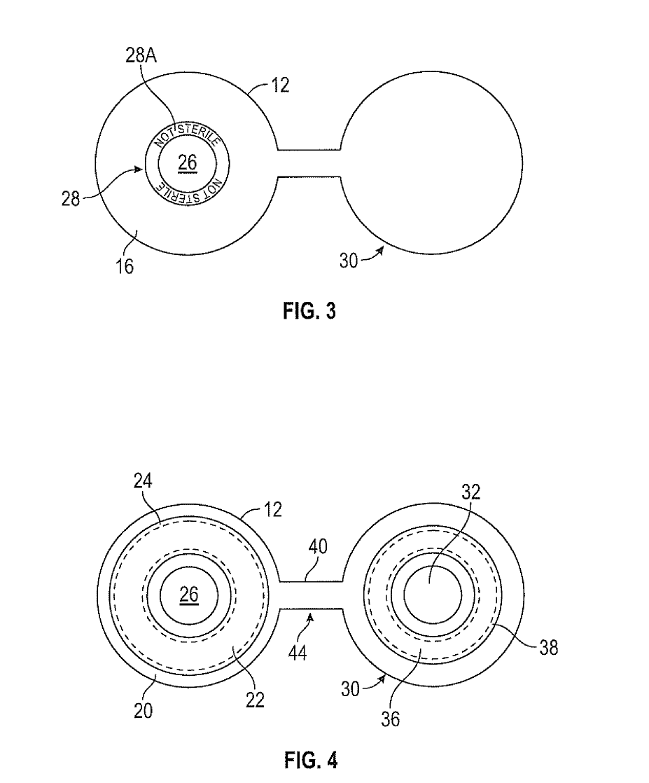

Referring to FIGS. 3-4, another embodiment of the bandage 10′ may be seen. The bandage 10′ may have a first section 12. In the present embodiment, the first section 12 may be circular in shape. The first section 12 may come in other shapes and/or sizes without departing from the spirit and scope of the present invention.

The first section 12 may be formed out a material that provides unidirectional elasticity and which “lifts” the skin of the patient thereby increasing blood flow and circulation. This may lead to less pressure on the nociceptors, or pain detectors, of the patient. This may theoretically decreases pain from injection of as needle as shown below. In accordance with one embodiment, the first section 12 may be formed out elastic therapeutic tape like kinesiology tape. This type of material is a thin, elastic cotton tape that can stretch up to 140% of its original length. As a result, if the tape is applied to a patient on a stretch greater than its normal length, the tape may “recoil” after being applied and therefore create a pulling “lifting” force on the skin.

The first section 12 may have a top surface 16 and a bottom surface 20. The bottom surface 20 may have an adhesive member 22 formed on opposing ends thereof. The adhesive members 22 may be used to attach the bandage 10 to the skin of the patient. A backing 24 may be applied over the adhesive members 22. The backing 24 may be removed before attaching the bandage 10 to the skin of the patient.

An opening 26 may be formed through the first section 12 of bandage 10. The opening 26 may be formed through a central area of the first section 12. The opening 26 may be used to indicate an injection area for administering a vaccination or insertion of a needle for other medical purposes into the patient. The opening 26 may remind the medical professional the area that needs to be and/or was sterilized. Without this indicator, the medical professional may swab and forget the exact area that was sterilized.

The area around the opening 26 may have an indicator 28 to show when the area in and around the opening 26 has been sterilized. The indicator 28 may be a chemical that indicates when the area in and around the opening 26 has been sterilized. In accordance with one embodiment, chromic acid, may be applied into the top layer of the area around the opening 26. The chemical reaction between the isopropyl alcohol that is impregnated in the alcohol swabs and the chromic acid turns the field around the opening 26 green. This may let the medical professional know that the injection site/field has been sterilized. Alternatively, the indicator 28 may be indicia 28A formed around the opening 26. The indicia 28A may be text such as “NOT STERILE” or the like, encircling the opening 26. When the isopropyl alcohol saturated alcohol pad is applied in and around the opening 26, the wording, “NOT STERILE” may change colors and/or may evaporate and disappear. This may remind the medical professional that the area in and around the opening 26 has or has not been swabbed prior to injection.

The bandage 10 may have a cover member 30. The cover member 30 may have a pad 32 formed on a first surface 34. The pad 32 may be formed of cotton gauze or like material. An adhesive 36 may be formed around a perimeter of the cover member 30. The adhesive 36 may be used to attach the cover member 30 over the opening 26. A backing 38 may be applied on the adhesive 36. The backing 38 may be removed from the adhesive 36 when the adhesive 36 may be used to attach the cover member 30 over the opening 26.

A retaining strip 40 may be used to attach the cover member 30 to the first section 12. The retaining strip 40 may be attached to the first section 12 and the cover member 30. The retaining strip 40 may form a “pull-tab” 44. The “pull-tab” 44 may be used for removing the bandage 10 by lifting on the “pull-tab” 44 up and away from the skin of the patient.

The foregoing description is illustrative of particular embodiments of the application, but is not meant to be a limitation upon the practice thereof. The following claims, including all equivalents thereof, are intended to define the scope of the application.

What is claimed is:

1. A bandage comprising:
a first section having a first section adhesive backing formed on a rear surface of the first section;
an opening formed through a central area of the first section;
an indicator formed on a front surface of the first section showing when an area around and in the opening has been sterilized; and
a cover member attached to the first section closing the opening.

2. The bandage of claim 1, comprising grip members formed on a top surface of the first section.

3. The bandage of claim 1, comprising a strip attaching the first section to the cover member.

4. The bandage of claim 1, wherein the strip attaching the first section to the cover member forms a tab member when the cover member is positioned over the opening to lift and remove the bandage.

5. The bandage of claim 1, comprising:
a pad attached to a rear surface of the cover member,
a cover adhesive applied around a perimeter of the pad; and
a cover backing applied to the cover adhesive.

6. The bandage of claim 1, wherein the indicator comprise a coating applied to an area around and in the opening, the coating changing color when an antiseptic is applied to the area around and in the opening.

7. The bandage of claim 1, wherein the indicator comprises indicia formed around the opening, wherein the indicia one of changes colors or disappears when an antiseptic is applied to the indicia.

8. The bandage of claim 1, comprising a first section backing applied to the first section adhesive backing.

9. A bandage comprising:
a first section having a first section adhesive backing formed on a rear surface of the first section;
an opening formed through a central area of the first section;
an indicator formed on a front surface of the first section showing when an area around and in the opening has been sterilized;
a cover member attached to the first section closing the opening; and
a strip attaching the first section to the cover member, wherein the strip attaching the first section to the cover member forms a tab member when the cover member is positioned over the opening to lift and remove the bandage.

10. The bandage of claim 9, comprising grip members formed on a top surface of the first section.

11. The bandage of claim 9, comprising:
a pad attached to a rear surface of the cover member;
a cover adhesive applied around a perimeter of the pad; and
a cover backing applied to the cover adhesive.

12. The bandage of claim 9, wherein the indicator comprise a coating applied to an area around and in the opening, the coating changing color when an antiseptic is applied to the area around and in the opening.

13. The bandage of claim 9, wherein the indicator comprises indicia formed around the opening, wherein the indicia one of changes colors or disappears when an antiseptic is applied to the indicia.

14. The bandage of claim 9, comprising a first section backing applied to the first section adhesive backing.

15. A bandage comprising:
a first section;
an opening formed through a central area of the first section;
a first section adhesive material applied to a rear section of the first section;
a first section backing applied to the first section adhesive material;
an indicator formed on a front surface of the first section showing when an area around and in the opening has been sterilized;
a cover member attached to the first section closing the opening;
a pad attached to a rear surface of the cover member,
a cover adhesive applied around a perimeter of the pad;
a cover backing applied to the cover adhesive; and
a strip attaching the first section to the cover member, wherein the strip attaching the first section to the cover member forms a tab member when the cover member is positioned over the opening to lift and remove the bandage.

16. The bandage of claim 15, comprising grip members formed on a top surface of the first section.

17. The bandage of claim 15, wherein the indicator comprise a coating applied to an area around and in the opening, the coating changing color when an antiseptic is applied to the area around and in the opening.

18. The bandage of claim 15, wherein the indicator comprises indicia formed around the opening, wherein the indicia one of changes colors or disappears when an antiseptic is applied to the indicia.
